(19) United States
(12) Patent Application Publication
(10) Pub. No.: US 2023/0057324 A1
(43) Pub. Date: Feb. 23, 2023

(54) Therapeutic Methods Using Bacterial Strains Which are Capable of Increasing Adenosine Levels

(71) Applicant: BioGaia AB, Stockholm (SE)

(72) Inventor: Stefan Roos, Uppsala (SE)

(73) Assignee: BioGaia AB, Stockholm (SE)

(21) Appl. No.: 17/251,410

(22) PCT Filed: Jul. 24, 2019
(86) PCT No.: PCT/EP2019/069987

§ 371 Date: Dec. 11, 2020

(30) Foreign Application Priority Data
Jul. 24, 2018 (GB) 1812079.0
Apr. 17, 2019 (GB) 1905470.9
Apr. 18, 2019 (GB) 1905591.2

(51) Int. Cl. A61K 35/747; A61P 17/02; A61K 31/7076; C12N 1/20; C12Q 1/44

(52) U.S. Cl. CPC A61K 35/747; A61P 17/02; A61K 31/7076; C12N 1/205; C12Q 1/44; A61K 2035/115

(57) ABSTRACT

The present invention relates to lactic acid bacterial strains which are capable of producing or inducing the production of adenosine, for use in the production of adenosine in a subject. Therapeutic uses of such strains include the treatment or prevention of diseases associated with adenosine deficiency, for example wound healing. Novel strains are also provided.

### FIELD OF THE INVENTION

The invention herein provides certain strains of lactic acid bacteria selected for their ability of increasing adenosine levels for prophylaxis and/or treatment of wounds (and other diseases as described elsewhere herein), a method of selecting such strains, and products containing such strains. Moreover this invention relates to preparations comprising substrate components being specifically chosen to enhance the efficacy of such strains.

### SEQUENCE LISTING

The present application is filed with a Sequence Listing in electronic format. The Sequence Listing file was created on Jan. 14, 2021, is entitled “2021-01-14_01189-0008 00US_Seq_List_ST25.txt,” and is 11,278 bytes in size. The information in the electronic format of the sequence listing is incorporated by reference herein in its entirety.

### BACKGROUND OF THE INVENTION

The Food and Agricultural Organization of the United Nations has defined probiotics as “live microorganisms which, when administered in adequate amounts, confer a health benefit on the host”. It is hypothesized that probiotics influence immune functions of the host, either via modulation of the microbiota composition, via metabolic activity, or even through a direct interaction with the immune system underlying the mucosa. Following such interaction, immune mechanisms may be activated as reflected by the release of immune mediators, such as cytokines, production of antibodies and activation of lymphocytes as well as other immune cells. These activated cells, cytokines and/or compounds released by the probiotics will exert immune modulatory functions at different location of the body through the blood circulation. Probiotics can also prevent or inhibit the proliferation of pathogens and suppress production of virulence factors by pathogens.

Several different bacterial strains are currently used as probiotics, including lactic acid producing bacteria, such as strains of Lactobacillus and Bifidobacteria. The effectiveness of probiotic bacteria is strain-specific, and each strain has its own mechanism by which a specific effect is mediated to improve health and to relieve symptoms of, for instance, gastrointestinal disturbance, including diarrhea and constipation, inflammatory bowel disease (IBD), irritable bowel syndrome (IBS) and colic but also skin disorders such as wounds.

One important metabolic process in the human body is purine metabolism, wherein purines are metabolized and broken down by specific enzymes. One example of such enzyme is ecto-5′-nucleotidase (CD73), which is considered to be a key enzyme in the generation of adenosine. Adenosine is a known immunosuppressive compound and functions mainly through four G protein-coupled receptors, A1, A2a, A2b and A3. Modulation of adenosine has been under investigation as a therapeutic approach in order to influence the immune and inflammatory systems. Extracellular adenosine concentrations from normal cells are approximately 300 nM; however, in response to cellular damage (e.g. in inflammatory or ischemic tissue), these concentrations are quickly elevated (600-1,200 nM). Thus, in regard to stress or injury, the function of adenosine is primarily that of cytoprotection preventing tissue damage during instances of hypoxia, ischemia, and seizure activity. Adenosine has an extremely short half-life so when used in clinical settings the mode of administration has to be carefully evaluated. Intravenous administration of adenosine is used when treating certain kinds of irregular heartbeat. Topical treatment of adenosine to foot wounds in diabetes mellitus has been shown in lab animals to drastically increase tissue repair and reconstruction.

Wound healing is a complex process, and is achieved through four overlapping phases: haemostasis, inflammation, proliferation and finally remodelling. The phases overlap and generally have a normal progression time of 4 weeks. A chronic wound has arrested in the inflammatory state and cannot progress further, for example cannot progress to the stages of proliferation and remodelling. Chronic wounds may thus never heal or may take years to do so. Such wounds cause patients severe emotional and physical stress and can lead to major disabilities and even in some cases death. They also create a significant financial burden on both the patients and the whole healthcare system. There is thus a need for improving and accelerating wound healing, as well as improving other conditions and/or diseases associated with or characterised by reduced, low or deficient adenosine production or synthesis or levels.

### SUMMARY OF THE INVENTION

As outlined above, wound healing is a complex process involving four overlapping phases: haemostasis, inflammation, proliferation and finally remodelling. Macrophages play key roles in all these phases. As wounds heal, the local macrophage population transitions from predominantly pro-inflammatory (M1-like phenotypes) to anti-inflammatory (M2-like phenotypes). Stimulation of the A2a receptor by adenosine in the presence of toll-like-receptor agonists switches macrophages from a pro-inflammatory M1-like phenotype into an anti-inflammatory/wound healing M2-like phenotype.

Adenosine has an extremely short half-life so when used in clinical settings the mode of administration has to be carefully evaluated. Intravenous administration of adenosine is used when treating certain kinds of irregular heartbeat, however recently the U.S. Food and Drug Administration (FDA) was warning health care professionals of the rare but serious risk of heart attack and death with such agents. Even if there are products on the market with less safety concerns reported there is still a need for better and more safer treatments using the benefits of the properties of adenosine.

The present invention is based on the finding that bacteria, for example lactic acid bacteria, can be selected which are capable of producing adenosine. Lactic acid bacteria producing adenosine have not been reported in the art.

Thus, the present invention provides a bacterial strain, e.g. a lactic acid bacterial strain which is capable of producing or inducing the production of adenosine, for use in the production of adenosine (e.g. increasing levels of adenosine or increasing or promoting the production of adenosine) in a subject.

Therapeutic uses of such adenosine producing bacterial strains can advantageously help address issues with the short half-life of adenosine. For example, such strains can continue to produce or induce the production of adenosine after they have been administered (for example allow continuous production), thereby providing a more long-term source of adenosine product. This property also provides clear advantages over the intravenous administration of adenosine which is proposed for some therapies, as here the adenosine will be rapidly cleared and further administration of adenosine will be required to replace it. In addition, the administration routes allowed by the use of such bacterial strains, e.g. probiotics, for example oral or rectal administration routes, readily allows frequent repeat dosing of subjects to be achieved, thereby improving the therapeutic effect. Another related advantage as described elsewhere herein is that it has been shown that strains that have the ability to produce or induce the production of adenosine can promote increased systemic levels of adenosine, even when the strains are administered orally rather than systemically, e.g. intravenously. This effect is particularly surprising and advantageous.

The present invention further provides a method for producing adenosine (e.g. increasing levels of adenosine or increasing or promoting the production of adenosine) in a subject, said method comprising the step of administering an effective amount of a bacterial strain, e.g. a lactic acid bacterial strain, which is capable of producing or inducing the production of adenosine, to said subject.

The present invention further provides the use of a bacterial strain, e.g. a lactic acid bacterial strain, which is capable of producing or inducing the production of adenosine, in the manufacture of a medicament or composition for use in the production of adenosine (e.g. increasing levels of adenosine or increasing or promoting the production of adenosine in a subject.

Such strains may be capable of producing or inducing the production of adenosine.

Such adenosine producing strains can be used in wound healing. In some cases it can be used in chronic wound healing. In other cases the wounds can be hard to heal, non-healing or severe wounds and in other aspects troubling. Any types of wounds can be treated using the present invention, for example any wounds affecting or damaging or disrupting the epithelium or endothelium of a subject. In particular, the types of wounds to be treated herein can be topical (e.g. on skin or other surface of a subject), oral and/or intestinal. Examples of wounds are burn wounds, venous and arterial ulcers, diabetic ulcers, pressure ulcers, pyoderma gangrenosum, recurrent aphthous stomatitis (ulcers) and wounds found in the intestine being part of the clinical manifestation of inflammatory bowel disease (IBD) such as ulcerative colitis and Crohn's disease (in other words intestinal wounds or wounds associated with IBD). Another example of a wound that can be treated by the invention herein is peptic ulcers. A further example of wounds that can be treated by the present invention are wounds in the oral cavity, for example oral or mouth ulcers, or aphthae. In all cases, in some aspects these wounds can be chronic wounds.

Without wishing to be bound by theory, it is believed that the production of adenosine, for example local or systemic production of adenosine, by these bacterial strains can be used to promote population transitions or phenotypic switching of macrophages from predominantly pro-inflammatory (M1-like phenotypes) to anti-inflammatory (M2-like phenotypes), for example to increase the number or proportion or percentage of macrophages with anti-inflammatory (M2-like phenotypes), for example compared to pro-inflammatory (M1-like phenotypes), and thereby facilitate or enable wound healing as described herein. Such increases would conveniently be assessed in comparison with a relevant control, for example in comparison with levels where no strain is present.

Such adenosine producing strains can thus equally be used for the treatment or prevention of any diseases associated with or characterised by reduced, low or deficient adenosine production or synthesis or levels in a subject. Some specific examples of diseases which can be treated in accordance with the invention are outlined below.

Adenosine is a natural ingredient used in cosmetics and personal care products that functions as a skin-restoring and soothing agent, as well as an anti-aging ingredient. One possible mechanism for adenosine's anti-wrinkle effect (and other relevant therapeutic or cosmetic effects), (Abella 2006, Int J Cosmet Sci, December; 28(6):447-51) is through collagen production. Adenosine may act at A2A and A2B adenosine receptors and thereby stimulate collagen matrix formation directly (Shaikh and Cronstein 2016, Purinergic Signalling 12(2), 191-197).

Adenosine has also been shown to promote thickening of hair on people with thinning hair. Studies have found that adenosine increases the anagen phase of hair growth and also increases the hair shaft diameter (Oura et al 2008, J. Dermatology, 35(12):763-7). Therefore, adenosine treatment may have significant results for hair growth and could help people suffering from androgenic alopecia.

Adenosine producing strains as described herein can thus be used in skin treatments (e.g. treatments which have a beneficial effect on the skin), for example can be used for restoring the skin (skin-restoring treatments) or for anti-aging treatments, such as an anti-wrinkle treatment. Such strains can also be used as a skin-soothing agent. Adenosine producing strains as described herein can also be used for hair treatments (e.g. treatments which have a beneficial effect on hair, for example head hair), for example can be used to promote or increase hair thickening or hair growth, or to treat or prevent hair loss, e.g. for types of alopecia such as androgenic alopecia.

Any appropriate mode of administration can be used for such treatments. In particular, topical and/or oral administration would be preferred for the treatment of the skin and hair conditions above.

Thus, in particular where hair or skin treatments are contemplated, the present invention also provides cosmetic uses as well as therapeutic uses. In some embodiments, cosmetic and therapeutic uses are combined. In other embodiments solely cosmetic uses (cosmetic use without therapeutic use) or solely therapeutic uses (therapeutic use without cosmetic use) are provided.

Reduced adenosine signaling and adenosine deficiency has been linked to several inflammatory conditions, including but not limited to colitis (Naganuma et al 2006, J Immunol, 177:2765-69, Kurtz et al 2014, Am J Physiol Gastrointest Liver Physiol, 307(3):G338-46), inflammatory bowel disease (Friedman et al 2009, PNAS, 106(39), 16788-93) and inflammatory bowel syndrome (Ochoa-Cortes et al 2014, Inflamm Bowel Dis, 20(7):1259-87). Adenosine producing strains as described herein can also be used in the treatment and/or prevention of inflammatory conditions. Preferably the strains may be used for treatment and/or prevention of inflammatory processes (or inflammatory diseases) in the gastrointestinal (GI) tract, genitourinary (GU) tract, oral cavity, in the lungs and airways, on the skin etc, of the mammalian body, including but not limited to colitis, inflammatory bowel disease (IBD), irritable bowel syndrome (IBS), diverticulitis, gingivitis, vaginitis etc.

Any appropriate mode of administration can be used for such treatments. In particular, oral administration would be conveniently used for the treatment of IBS, IBD, diverticulitis or other diseases affecting the GI tract. Oral and/or topical administration would be conveniently used for the treatment of vaginitis, gingivitis, or other diseases affecting the GU tract or oral cavity.

Regulatory T-cells (Tregs) are a subpopulation of T cells which modulate the immune system, maintain tolerance to self-antigens, and abrogate autoimmune disease. Tregs generally suppress or down regulate induction and proliferation of effector T cells including TH1, TH2, and TH17 subsets of T cells; these proinflammatory families of T cells are controlled via the interaction of adenosine (produced by Tregs) and the receptor A2A, which is highly expressed on T cells (Ohta and Sitkovsky 2014, Frontiers in Immunology, 10; 5:304).

Adenosine producing strains as described herein can thus be used in the treatment or prevention of a disease associated with Treg deficiency or Treg dysfunction.

Adenosine producing strains as described herein can therefore also be used in the treatment and/or prevention of autoimmune diseases, e.g. IPEX syndrome (immunodysregulation, polyendocrinopathy, and enteropathy, with X-linked inheritance).

Again, appropriate modes of administration can readily be determined depending on the disease in question.

Other objects and advantages of the present invention will become obvious to the reader and it is intended that these objects and advantages are within the scope of the present invention.

### BRIEF DESCRIPTION OF THE DRAWINGS

FIG. 1 shows the measurement of 5′-nucleotidase activity in the supernatant of L. reuteri bacterial cells.

FIG. 2 shows plasma adenosine in a mouse model as detected by metabolomic analysis. The comparisons of 3 groups as indicated. #: WT+LR vs. WT+MRS, #p<0.05; **: WT+LR vs. WT no treatment, ** p<0.01. n=8 mice per group.

FIG. 3A shows the measurement of 5′-nucleotidase activity in the supernatant of L. reuteri DSM 33198 bacterial cells as compared to DSM 17938 bacterial cells. FIG. 3B shows the measurement of 5′-nucleotidase activity in the supernatant of various L. reuteri bacterial cells as compared to DSM 17938 bacterial cells.

### DETAILED DESCRIPTION OF THE INVENTION AND PREFERRED EMBODIMENTS THEREOF

The wound healing process is achieved through four overlapping phases: haemostasis, inflammation, proliferation and finally remodelling. The phases overlap and generally have a normal progression time of 4 weeks, although in some cases wound healing can take more weeks or several months. A chronic wound has arrested in the inflammatory state and cannot progress further, for example cannot progress to the stages of proliferation and remodelling.

In wounds on the skin, platelet aggregation and release of chemokines and growth factors (such as IL-1, TNF-a and TGF-b) activate the local fibroblasts and keratinocytes and the immune cascade to initiate inflammation. Keratinocytes, as the major cell type of the epidermis, release proinflammatory cytokines (IL-8 and IL-6) and antimicrobial peptides and proteins. Together, this immune response is aimed at decontaminating the wound bed by recruitment and activation of macrophages, neutrophils, and keratinocytes that leads to sterilisation of the wound and subsequently to resolution of inflammation. Meanwhile, skin-resident T cells on the one hand and infiltrating T cells on the other hand participate in the inflammatory stage through production of IL-17, IL-22, and TNF-a further amplifying the host defence response. And finally, macrophages (M1-like phenotype) undergo a phenotypic switch from a key inflammatory and microbicidal player to anti-inflammatory and regulatory activities (M2-like phenotype). Angiogenesis is initiated and M2 macrophages activate fibroblasts to produce cytokines and growth factors (IL-10, VEGF and TGF-b) that stimulate wound closure by migration of keratinocytes.

In chronic wounds it is believed that pro-inflammatory macrophages (M1-like phenotype) are unable to phagocytise spent neutrophils and fail to switch to anti-inflammatory M2-like phenotype macrophages. This leads to the recruitment of more macrophages that continue to produce pro-inflammatory chemokines (IL-8) and cytokines (TNF-a) that fuel the inflammation and inhibit wound closure.

Stimulation of the A2A receptor by adenosine in the presence of toll-like-receptor agonists has been shown to switch macrophages from a pro-inflammatory M1-like phenotype into an anti-inflammatory/wound healing M2-like phenotype (Ferrante et al 2013, Inflammation, 36(4):921-31). Pro-inflammatory M1-like phenotype macrophages decrease both the expression and activity of CD39 and CD73 (considered to be a key enzyme in the generation of adenosine), leading to reduced ATP degradation. By contrast, M2-like phenotype macrophages, which are characterized by the production of anti-inflammatory cytokines (IL-10 and IL-1 receptor antagonist) and tissue remodeling molecules, showed an increased expression and activity of both catabolic enzymes, and were able to convert ATP into adenosine (Zanin et al 2012, PloS One, 7(2),e31205). Thus, M2 macrophages generate an adenosine-rich environment, which in turn can augment the anti-inflammatory and tissue remodeling activities of these cells.

Severe mucosal tissue damage requiring efficient wound healing is a main feature of inflammatory bowel disease (IBD), with its two entities Crohn's disease (CD) and ulcerative colitis (UC). Lamina propria monocytes and M1 macrophages disrupt the intestinal epithelial barrier through deregulation of tight junction proteins and induction of epithelial cell junction proteins and induction of epithelial cell apoptosis, induction of TNF-a, IL-1b and IL-18 thus driving intestinal inflammation in IBD. Adenosine can bind to the A2A receptor on M1 macrophages and induce a shift to M2 anti-inflammatory phenotype. Both UC and CD show a large number of pro-inflammatory M1 reside in the inflamed mucosa (Lissner et al 2015, Inflamm Bowel Dis, 21(6):1297-305), so an ability to shift these M1 macrophages to M2 macrophages, for example using the adenosine producing strains in accordance with the present invention, could aid wound healing in these diseases.

As outlined above, a preferred lactic acid bacterial strain is one which can produce or induce adenosine in a subject, particularly in the gastrointestinal (GI) tract, skin/wounds, oral tract/mouth, or other appropriate location depending on the disease in question. Such adenosine can act on appropriate cell surface adenosine receptors (for example A2A receptors, A2B receptors, A3 or A4 receptors, preferably A2A receptors) and result in the production of elevated levels of intracellular cAMP. A2A and other adenosine receptors are known to be present in cells found in the GI tract and cells that are part of the inflammatory processes for example. The adenosine should thus preferably be provided extracellularly in order to be able to bind to cell surface adenosine receptors. To this end, the adenosine may be produced within the bacterial cells and be transported extracellularly (or secreted). Alternatively, the adenosine may be produced extracellularly, for example on the bacterial cell surface or in the supernatant. Such extracellular production can for example conveniently take place by way of the presence of a cell wall (or cell surface) anchored 5′-nucleotidase enzyme (or ecto 5′-nucleotidase enzyme, CD73), which can convert appropriate substrate to adenosine. Such extracellular production can equally take place by way of the presence of a 5′-nucleotidase enzyme (or ecto 5′-nucleotidase enzyme) in the cell supernatant or extracellular space. As shown herein, bacterial strains can provide or produce or release such a 5′-nucleotidase enzyme (or ecto 5′-nucleotidase enzyme) into the cell supernatant or extracellular space where it can then convert appropriate substrate to adenosine. Appropriate substrates include AMP.

Thus, when bacterial strains are referred to herein as being capable of producing adenosine, this includes the direct production of adenosine by the bacterial cells themselves and also includes the production of adenosine by the bacterial cells by way of the cells having an active 5′-nucleotidase enzyme (for example present on the bacterial cell surface or released into the cell supernatant or extracellular space) and thereby converting or being able to convert appropriate substrate, e.g. AMP, to adenosine. Such substrate can be naturally present, e.g. endogenously in the environment, or can be provided to the bacteria, e.g. by exogenous means.

Thus, the strains of the invention can be used to treat any disease or condition associated with (or characterised by) reduced or decreased levels of or adenosine, or associated with (or characterised by) adenosine deficiency, or can be used to treat any disease or condition which would benefit from increased levels of adenosine. Such diseases or conditions (and indeed subjects suffering from such diseases or conditions) would be readily recognised by those skilled in the art and would for example include diseases, conditions or subjects which have low, reduced e.g. significantly reduced (or abnormal) levels of adenosine or adenosine deficiency, e.g. compared to levels in a normal or healthy subject, for example levels in a normal or healthy subject of the same, equivalent or comparable age. A preferred example of such a disease is wounds. In some cases it can be used in chronic wound healing. In other cases the wounds can be hard to heal, non-healing or severe wounds and in other aspects troubling. Any types of wounds can be treated using the present invention, for example any wounds affecting or damaging or disrupting the epithelium or endothelium of a subject. In particular, the types of wounds to be treated herein can be topical (e.g. on skin or other surface of a subject), oral and/or intestinal. Examples of wounds are burn wounds, venous and arterial ulcers, diabetic ulcers, pressure ulcers, pyoderma gangrenosum, recurrent aphthous stomatitis (ulcers) and wounds found in the intestine being part of the clinical manifestation of inflammatory bowel disease (IBD) such as ulcerative colitis and Crohn's disease (in other words intestinal wounds or wounds associated with IBD). Another example of a wound that can be treated by the invention herein is peptic ulcers. A further example of wounds that can be treated by the present invention are wounds in the oral cavity, for example oral or mouth ulcers, or aphthae. In all cases, in some aspects these wounds can be chronic wounds.

Other examples of diseases to be treated are described elsewhere herein. For example, diseases or conditions to be treated include wound healing, skin treatments, hair treatments, inflammatory conditions, diseases associated with Treg deficiency or Treg dysfunction, or autoimmune diseases. In particular, diseases or conditions to be treated include topical, oral and/or intestinal wounds, skin restoring treatments, anti-ageing treatments, treatments to promote or increase hair thickening or hair growth, to treat or prevent hair loss, to treat or prevent inflammatory processes in the gastrointestinal tract, genitourinary tract, oral cavity, in the lungs and/or airways, or on the skin, or to treat or prevent IPEX syndrome.

Preferred strains for such therapeutic uses are those which produce adenosine.

Thus, yet further aspects of the invention provide a lactic acid bacterial strain, or a Lactobacillus strain, which is capable of producing or inducing the production of adenosine. Therapeutic uses of such strains are also provided. Preferably, said strains have a gene encoding a 5′-nucleotidase or such strains have an active 5′-nucleotidase enzyme, for example to convert AMP (or other appropriate substrate) to adenosine. Exemplary levels of adenosine production or levels of 5′-nucleotidase activity are described elsewhere herein.

Preferred strains of the present invention are L. reuteri strains, more preferably L. reuteri strains DSM 32846, DSM 32847, DSM 32848, DSM 32849 and/or DSM 33198 (the depositary details of which are provided elsewhere herein), and such strains, e.g. isolated strains or biologically pure cultures or preparations of such strains, form further aspects of the invention, as do compositions (e.g. pharmaceutical or nutritional, e.g. food supplements, or probiotic, compositions, e.g. with pharmaceutically or nutritionally acceptable diluents and/or excipients) comprising said strains, or the therapeutic use of such strains, for example as described elsewhere herein, in particular for the treatment or prevention of wounds, e.g. chronic wounds, or other diseases as described elsewhere herein. Another preferred strain for the therapeutic uses described herein is DSM 17938 (the depositary details of which are provided elsewhere herein). However, in some embodiments the DSM 17938 strain is not used. Thus, the present invention provides the Lactobacillus reuteri strains DSM 32846, DSM 32847, DSM 32848, DSM 32849 and/or DSM 33198 for use in therapy, for example for the treatment or prevention of diseases as described elsewhere herein. The present invention further provides the Lactobacillus reuteri strains DSM 32846, DSM 32847, DSM 32848, DSM 32849 and/or DSM 33198 for use in the treatment or prevention of wounds, e.g. chronic wounds, as described elsewhere herein, or in the treatment or prevention of other diseases as described elsewhere herein. The present invention further provides the Lactobacillus reuteri strain DSM 17938 for use in the treatment or prevention of wounds, e.g. chronic wounds, as described elsewhere herein, or in the treatment or prevention of other diseases as described elsewhere herein.

Thus, a yet further aspect of the invention provides a method of treatment or prevention of wounds, e.g. chronic wounds, or other diseases as described elsewhere herein, in a subject, said method comprising the step of administering an effective amount of the Lactobacillus reuteri strain DSM 32846, DSM 32847, DSM 32848, DSM 32849 and/or DSM 33198, to said subject. Lactobacillus reuteri strain DSM 17938 is also preferred in some embodiments.

In another aspect, the present invention provides the use of the Lactobacillus reuteri strains DSM 32846, DSM 32847, DSM 32848, DSM 32849 and/or DSM 33198 in the manufacture of a medicament or composition for use in the treatment or prevention of wounds, e.g. chronic wounds, or other diseases as described elsewhere herein, in a subject. Lactobacillus reuteri strain DSM 17938 is also preferred in some embodiments.

Alternative and preferred embodiments and features of the invention as described elsewhere herein apply equally to the methods of treatment and uses of the invention described here and elsewhere herein.

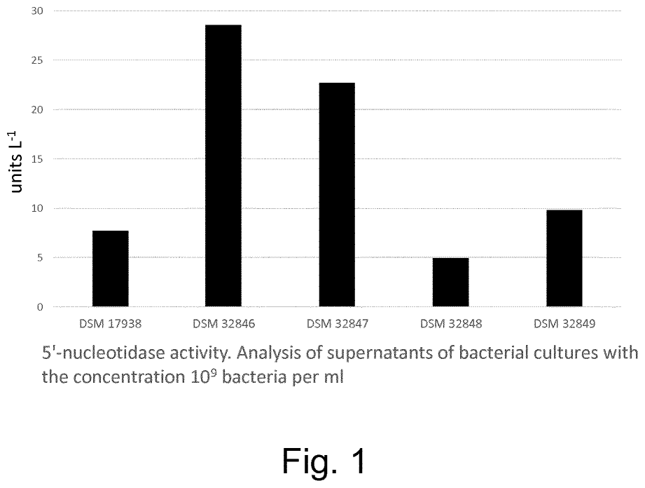

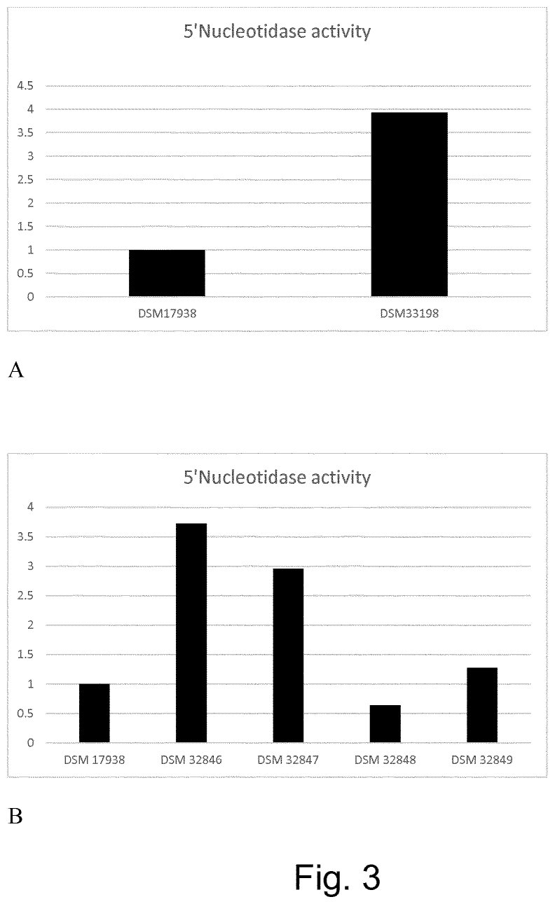

The strains L. reuteri DSM 32846, DSM 32847, DSM 32848, DSM 32849 and/or DSM 33198 have been selected for their capability to produce adenosine, for example by having a gene encoding a 5′-nucleotidase, and an active 5′-nucleotidase enzyme which can convert AMP substrate to adenosine, and are suitable to increase levels of adenosine (e.g. in comparison to levels where the strains are not present). The strains comprise an active cell wall anchored 5′-nucleotidase enzyme and also show 5′-nucleotidase enzyme activity in their respective supernatants (see FIG. 1 and FIG. 3A/B). Lactobacillus reuteri strain DSM 17938 also has this capability.

These strains of L. reuteri have been developed (in other words are modified or adapted or evolved) from naturally occurring strains and have also been selected for one or more other improved properties such as increased resistance to bile or increased adherence to mucosal surfaces, e.g. surfaces of the GI tract. Thus, DSM 32846 and DSM 32847 have been evolved to be more tolerant to bile acids and thereby for example survive in larger numbers in the GI-tract. DSM 32848 and DSM 32849 have been evolved to adhere better to mucus, with the aim to colonize better in the GI-tract and thereby function better for example according to the present invention. The L. reuteri strain DSM 33198 has also been modified in a multi-step selection process including a repeated freeze-drying procedure to allow it to be more tolerant and give a higher survival in the production process than its native isolate (parent strain). Thus, such strains do not correspond to strains occurring in nature and have been forced to evolve and are non-native strains. All strains Lactobacillus reuteri DSM 32846, DSM 32847, DSM 32848, DSM 32849 and DSM 33198 are selected or used. Preferred strains are DSM 32846, DSM 32847, DSM 32849 or DSM 33198. More preferred strains are DSM 32846, DSM 32847 or DSM 33198. In one particular aspect of the invention the strains are DSM 32846 and/or DSM 32847, for example DSM 32846 is preferred in some embodiments. In some embodiments, strain DSM 33198 is selected or used.

Thus, the present invention also provides a lactic acid bacterial strain which is capable of producing or inducing the production of adenosine (e.g. increasing levels of adenosine, or increasing or promoting the production of adenosine), for use in the production of adenosine in a subject, for example systemic or local production in a subject. Such lactic acid bacterial strain preferably has a gene encoding a 5′-nucleotidase, e.g. a cell wall anchored 5′-nucleotidase, or has an active 5′-nucleotidase enzyme, e.g a cell wall anchored 5′-nucleotidase enzyme. Such strains can be used in the treatment or prevention of a disease associated with (or characterised by) a deficiency or reduced levels of adenosine, for example as compared to levels in an equivalent healthy subject, or can be used to treat or prevent any disease or condition which would benefit from increased levels of adenosine.

Thus, the present invention further provides a method for producing adenosine in a subject, said method comprising the step of administering an effective amount of a bacterial strain, e.g. a lactic acid bacterial strain, which is capable of producing or inducing the production of adenosine (e.g. increasing levels of adenosine, or increasing or promoting the production of adenosine), to said subject.

The present invention further provides the use of a bacterial strain, e.g. a lactic acid bacterial strain, which is capable of producing or inducing the production of adenosine (e.g. increasing levels of adenosine, or increasing or promoting the production of adenosine), in the manufacture of a medicament or composition for use in the production of adenosine in a subject.

Adenosine increases intracellular cAMP levels via adenosine receptor.

It is a further object of the invention to provide new bacterial strains, which are capable of producing adenosine.

One important metabolic process in the human body is purine metabolism, wherein purines are metabolized and broken down by specific enzymes. One example of those enzymes are ecto-5′-nucleotidase (CD73), which is considered to be a key enzyme in the generation of adenosine.

The inventors surprisingly found that some specific probiotic bacterial strains were capable of producing adenosine.

The present invention thus includes a new method of selecting specific bacterial strains, including strains of lactic acid bacteria, that are effective in producing adenosine. The purpose of selecting specific bacterial strains is to use them for treating certain disorders such as wounds, e.g. chronic wounds, or for treating other diseases as described elsewhere herein.

The present invention provides a method for selecting bacterial strains, in particular lactic acid bacterial strains, which are useful as probiotics and in therapy, e.g. as pharmaceuticals or as food supplements.

One aspect of the present invention thus provides a method for the selection of a bacterial strain, preferably a lactic acid bacterial strain, capable of producing adenosine, wherein said method comprises:
a) screening a bacterial strain, e.g. a lactic acid bacterial strain, for the presence of a gene encoding a 5′-nucleotidase, e.g. a cell wall anchored 5′-nucleotidase; and/or
b) screening a bacterial strain, e.g. a lactic acid bacterial strain, or its supernatant for the presence of an active 5′-nucleotidase enzyme, e.g. a cell wall anchored 5′-nucleotidase enzyme.

Such 5′-nucleotidase (5′NT) enzymes can also be referred to as ecto-5′ nucleotidase enzymes or CD73 (cluster of differentiation 73). Strains which are capable of producing adenosine can then be selected.

Viewed alternatively, the present invention provides a method for the selection of a bacterial strain, preferably a lactic acid bacterial strain, by screening a bacterial strain, e.g. a lactic acid bacterial strain, for its capability to produce adenosine and selecting a strain which has that capability.

Once an appropriate strain has been selected using the method of the present invention it can then be used for the production, e.g. the local or systemic production, of adenosine in a subject. Said strains thus also have to be capable of production, e.g. the local or systemic production, of adenosine in a subject.

A bacterial strain, e.g. a lactic acid bacterial strain, selected, produced, obtained or obtainable by the methods of the invention, wherein said strain is capable of producing adenosine, for example local or systemic production of adenosine in a subject, is a yet further aspect of the invention.

Therapeutic uses in wounds, e.g. chronic wounds, or in other diseases as described elsewhere herein, of the strains selected by the present invention are also provided.

Thus, a further aspect of the present invention provides a bacterial strain, e.g. a lactic acid bacterial strain, selected, produced, obtained or obtainable by the method of the invention, wherein said strain has a 5′-nucleotidase gene or an active 5′-nucleotidase enzyme and is capable of producing adenosine, for use in the production, e.g. local or systemic production, of adenosine in a subject.

Alternative embodiments of the invention provide a bacterial strain, e.g. a lactic acid bacterial strain, wherein said strain has a 5′-nucleotidase gene or an active 5′-nucleotidase enzyme and is capable of producing adenosine, for use in the production, e.g. local or systemic production, of adenosine in a subject. Preferred features of this strain and its uses are described elsewhere herein.

Methods of treatment or methods for the production, e.g. local or systemic production, of adenosine in a subject, are also provided, said methods comprising the administration of a bacterial strain, e.g. a lactic acid bacterial strain, selected, produced, obtained or obtainable by the selection method of the invention, or the administration of a bacterial strain, e.g. a lactic acid bacterial strain, wherein said strain has a 5′nucleotidase gene or an active 5′nucleotidase enzyme and is capable of producing adenosine, to said subject in an amount effective to enable production, e.g. local or systemic production, of adenosine in said subject. Preferred features of the strain and its therapeutic uses, e.g. in wounds, e.g. chronic wounds, or in the treatment or prevention of other diseases as described elsewhere herein, are described elsewhere herein.

Also provided by the present invention is the use of a bacterial strain, e.g. a lactic acid bacterial strain, selected, produced, obtained or obtainable by the selection method of the invention, wherein said strain has a 5′nucleotidase gene or an active 5′nucleotidase enzyme and is capable of producing adenosine, in the manufacture of a composition or medicament for use in the production, e.g. local or systemic production, of adenosine in a subject. Alternative embodiments provide the use of a bacterial strain, e.g. a lactic acid bacterial strain, wherein said strain has a 5′nucleotidase gene or an active 5′nucleotidase enzyme and is capable of producing adenosine, in the manufacture of a composition or medicament for use in the production, e.g. local or systemic production, of adenosine in a subject. Preferred features of the strain and its therapeutic uses, e.g. in wounds, e.g. chronic wounds, or in the treatment or prevention of other diseases as described elsewhere herein, are described elsewhere herein.

Products or compositions, e.g. pharmaceutical compositions, probiotic compositions, or dietary/nutraceutical compositions, comprising the bacterial strains (for example comprising one or more of the bacterial strains) as described herein (e.g. bacterial strains capable of producing or inducing adenosine, etc.) and uses of said products or compositions in methods and uses as described herein form yet further aspects of the invention.

The Food and Agricultural Organization of the United Nations define probiotics as “live microorganisms which when administered in adequate amounts confer a health benefit on the host”. Nowadays, a number of different bacteria are used as probiotics for example, lactic-acid producing bacteria such as strains of Lactobacillus and Bifidobacterium.

Alternative and preferred embodiments and features of the invention as described elsewhere herein apply equally to the methods of treatment, uses and products of the invention.

As described above, the present invention relates to the selection and use of bacterial strains that are capable of producing adenosine.

Said strains which are effective in producing adenosine can be used for local or systemic production of adenosine in a subject, e.g. a mammal, preferably a human.

Thus, as outlined above, the present invention provides various methods for the selection or screening of bacterial strains capable of producing adenosine.

Some of the methods comprise a step (e.g. step a)) of screening for the presence of a gene encoding a 5′-nucleotidase, e.g. a cell wall anchored 5′-nucleotidase. Such screening can be carried out using any appropriate method and strains positive for a 5′-nucleotidase gene are selected. Such methods will conveniently be carried out in vitro, for example will be genetic or nucleic-acid based methods to detect the presence of a gene encoding a 5′nucleotidase, e.g. a cell wall anchored 5′nucleotidase (or an identificatory fragment thereof) the sequences of which are known in the art. For example, a PCR protocol (or other nucleic acid based technique) can be readily designed to do this based on the known nucleic acid sequences encoding the enzymes, or as an alternative, genome sequences of candidate strains could be scrutinized with the aim to identify the said gene encoding a 5′nucleotidase, e.g. a cell wall anchored 5′nucleotidase, for example, based on homology to known 5′nucleotidase sequences, including for example the presence of an LPXTG-motif (SEQ ID NO:1) as discussed below.

An exemplary gene to be detected in the methods of the invention is the cell wall anchored (ecto) 5′nucleotidase gene from L. reuteri (e.g. GenBank accession number: AEI56270.1, LPXTG-motif cell wall anchor domain protein [Lactobacillus reuteri SD2112]), or an appropriate homologue/5′ nucleotidase from other species of bacteria, e.g. other lactic acid bacteria. An exemplary technique is described in the experimental Examples.

Some methods comprise a step (e.g. step b)) of screening for the presence of an active (functional) 5′-nucleotidase enzyme, e.g. a cell wall anchored 5′nucleotidase enzyme. Such a step can be carried out using any appropriate method, for example, using an enzymatic assay. The 5′-nucleotidase enzyme catalyzes the following reaction: AMP+H₂O⇄adenosine+phosphate (AMP is adenosine monophosphate) and an assay to measure this reaction can readily be used to determine the presence of an active 5′-nucleotidase enzyme (or 5′-nucleotidase activity). In other words the reference herein to an active or functional 5′-nucleotidase enzyme is one which is capable of catalyzing this reaction under suitable conditions, e.g. when supplied with appropriate substrate such as AMP. The activity of the 5′-nucleotidase enzyme can, if desired, be quantified, e.g. in such an enzymatic assay, for example by measuring the amount or concentration of phosphate or adenosine or other appropriate downstream product of adenosine which is generated in the reaction and can be measured or quantitated. The activity can conveniently be measured on a sample of the bacterial cells, or the supernatant from the bacterial cells.

Methods for carrying out such an assay would be well-known to a person skilled in the art. For example, appropriate kits are commercially available, such as the Crystal Chem 5′-Nucleotidase Assay Kit (Crystal Chem, catalogue #80229, Downers Grove, Ill., USA) in which 5′-nucleotidase enzyme activity is measured by way of the production of a dye (a quinone dye) which is formed as a downstream product of the conversion of AMP to adenosine (i.e. as a result of the production of adenosine). AMP is provided as a substrate. An exemplary technique is described in the Examples section. Appropriate methodology is also readily available from other reagent suppliers, e.g. Sigma, in conjunction with their 5′-nucleotidase enzyme reagent.

Strains with high or significant production levels of 5′-nucleotidase enzyme (or 5′-nucleotidase activity) are preferred, for example strains that have a 5′-nucleotidase activity of at least or greater than 2 units/L (units per litre) and/or are capable of producing adenosine at a level of at least or greater than 2 μmol L⁻¹ min⁻¹ (μmol per litre per minute). Thus, in preferred embodiments, a strain is selected for having 5′-nucleotidase activity at a level of at least, or greater than, 3, 4, 5, 8, 9, 10, 15, 20, 25, 30, 35, 40, 45, 50, 60, 70, 80, 90, 100, 110, 120, 130, 140 or 150 units/L, and/or is capable of producing adenosine at a level of at least, or greater than, 3, 4, 5, 8, 9, 10, 15, 20, 25, 30, 35, 40, 45, 50, 60, 70, 80, 90, 100, 110, 120, 130, 140 or 150 μmol L⁻¹ min⁻¹. In some embodiments such values can represent upper limits. Such values generally refer to values of 5′-nucleotidase activity measured on a sample of the bacterial cells, e.g. on the surface of the bacterial cells, or in the supernatant of the bacterial cells in culture, preferably in the supernatant. Such values generally refer to adenosine levels (preferably extracellular adenosine levels) measured in a sample of the bacterial cells, e.g. in the supernatant of the bacterial cells in culture. Such values generally refer to values of 5′-nucleotidase activity (or adenosine levels) when measured with a concentration of 10⁹ bacteria/ml, or the supernatant from such a culture. One unit is defined as the amount of enzyme needed for producing 1 μmol product/min (e.g. 1 μmol product/litre/min). The concentration in units/liter will then correspond to the enzyme concentration needed for increasing the concentration with 1 μM/min (e.g. 1 μM/litre/min). An appropriate and exemplified assay for measuring this activity is shown in the Examples using the 5′-nucleotidase kit (#80229) from Crystal Chem Inc. Thus, in preferred embodiments the above units and values refer to units and values when measured using this kit and/or the conditions set out in the Examples, in particular with a concentration of 10⁹ bacteria/ml, or the supernatant from such a culture, or an equivalent assay. Thus, such methods will conveniently be carried out in vitro.

Methods comprising at least step b) will be preferred, as the presence of a gene encoding a 5′-nucleotidase is not always indicative of the presence of an active 5′-nucleotidase enzyme. In the methods, uses and lactic acid bacteria of the invention, adenosine production or adenosine activity should take place extracellularly, i.e. outside or on the surface of the lactic acid bacteria, so that it can for example be present in the supernatant or other extracellular fluid produced by the lactic acid bacteria. Thus, the presence of an active 5′-nucleotidase enzyme on the cell surface, for example in the form of a cell wall anchored 5′-nucleotidase, or extracellular from the bacterial cell, for example in the supernatant, can be a useful feature such that the reaction to produce adenosine takes place outside the cell, for example on the surface of the bacterial cell.

Thus, the production of good levels of adenosine (e.g. extracellular adenosine produced by the 5′-nucleotidase enzyme) can also be an indicator of the presence of a 5′-nucleotidase gene or the presence of an active 5′-nucleotidase enzyme. Thus, the selection method of the invention can also involve the step of selecting a strain which is producing adenosine. Strains with high or significant production levels of adenosine, e.g. extracellular adenosine, are preferred, for example strains that produce adenosine at a level which is therapeutically effective in a subject. Such values generally refer to values of adenosine measured in the supernatant of strains in culture or on the cell surface of strains in culture (in vitro) and some exemplary specific values are provided above.

However, in some embodiments it is appropriate to refer to values, levels or amounts of adenosine produced in a subject, for example locally, e.g. at the site of administration, e.g. in the gastrointestinal tract, or systemically, e.g. in the blood or plasma. For example, preferred strains can enable increased production of adenosine, for example increased in vivo production, e.g. local (e.g. in the GI tract) or systemic production, of adenosine, for example when compared to a relevant control as described elsewhere herein, such as the levels of adenosine when no strains are administered, or the base or natural level of adenosine in a particular subject.

In all aspects of the invention described herein, local production can refer to production or levels of adenosine at the site of administration, for example production or levels of adenosine in the gastrointestinal tract (e.g. when administration is oral). Systemic production may conveniently refer to production or levels of adenosine in the circulatory system, for example in blood or plasma. In particular, the Examples herein show that strains which are capable of producing or inducing the production of adenosine in vitro/in culture, can also be capable of producing or inducing the production of adenosine in vivo, for example by promoting or inducing increased systemic levels of adenosine, for example in plasma or blood. Such increased systemic levels of adenosine were even observed when strains were administered orally. The ability of strains to stimulate or promote increased systemic levels of adenosine in this way, in particular when the strains are administered orally rather than systemically, e.g. intravenously, is particularly surprising and advantageous.

Strains which can give rise to increased systemic levels of adenosine (or 5′ nucleotidase activity), for example in the plasma or blood of a subject, are thus preferred, in particular where such an effect is observed when the strains are administered orally. Preferably said increases are measurable or significant increases, for example are statistically or clinically significant. By way of example, strains which can give rise to increases of at least, or up to, 1.5 fold, 2 fold, 2.5 fold, 3 fold, 3.5 fold, 4 fold, 4.5 fold, 5 fold, 6 fold, 7 fold, 8 fold, 9 fold or 10 fold, in levels of adenosine (or 5′ nucleotidase activity), for example local or systemic levels of adenosine (or 5′ nucleotidase activity), preferably blood or plasma adenosine, are preferred. Any appropriate comparison can be used, for example an increase when compared to the levels observed when no strain is administered, or the levels when a control formulation, e.g. a control formulation not containing the relevant strain, is administered.

Appropriate methods of measuring levels of adenosine (or 5′ nucleotidase activity) production would be well known to a person skilled in the art. Thus, in some embodiments of the invention, the selection method will involve the step of detecting or determining the amount or level (e.g. the concentration) of adenosine produced by a candidate strain.

Optionally, the levels of adenosine production or 5′nucleotidase activity (or indeed any other appropriate property of the strains described herein) can conveniently be compared to positive or negative control strains. An appropriate positive control strain might be DSM 17938 which has been shown in the Examples to produce significant levels of adenosine/5′nucleotidase activity, for example in the supernatant of bacterial cells. Some strains will produce higher levels, sometimes significantly higher levels, of adenosine/5′nucleotidase activity than DSM 17938, for example in the supernatant of bacterial cells. Thus, strains capable of producing higher (increased) levels, or significantly higher (increased) levels, of 5′nucleotidase activity than DSM 17938, for example in the supernatant of bacterial cells, for example when assessed in vitro, form a yet further aspect of the invention. Exemplary strains are DSM 32846, DSM 32847 and DSM 32849 (see FIG. 1) as well as DSM 33198 (see FIG. 3A). Viewed alternatively, strains capable of producing or inducing production of higher (increased) levels, or significantly higher (increased) levels, of adenosine than DSM 17938, for example in the supernatant of bacterial cells, for example when assessed in vitro, form a yet further aspect of the invention. For example, strains capable of producing or inducing production of higher (increased) levels, or significantly higher (increased) levels of adenosine in a subject, for example higher etc., local or systemic, e.g. plasma or blood, levels of adenosine in a subject than DSM 17938, for example when assessed in vivo, form a yet further aspect of the invention, in particular where such an effect is observed when the strains are administered orally.

In general, strains which have one or more improved (e.g. significantly improved) properties when compared to DSM17938 are preferred for some embodiments.

Preferably said increases (in adenosine production or 5′nucleotidase activity) are measurable or significant increases, for example are statistically or clinically significant. By way of example, strains which can give rise to increases of at least, or up to 10%, 20%, 30%, 40%, 50%, 60%, 70%, 80%, 90%, 100%, 150%, 200%, 250%, 300%, 350%, 400%, 450%, 500%, 550%, or higher, in levels of adenosine, for example local or systemic levels of adenosine, preferably plasma or blood adenosine, or in vitro levels of adenosine, or in levels of 5′nucleotidase activity (for example when assessed in vitro), compared to the levels with DSM 17938 are preferred. Viewed alternatively, strains which can give rise to increases of at least, or up to, 1.5 fold, 2 fold, 2.5 fold, 3 fold, 3.5 fold, 4 fold, 4.5 fold, 5 fold, 6 fold, 7 fold, 8 fold, 9 fold or 10 fold, in levels of adenosine, for example local or systemic levels of adenosine, preferably plasma or blood adenosine, or in vitro levels of adenosine, or in levels of 5′nucleotidase activity (for example when assessed in vitro), compared to the levels with DSM 17938 are preferred. In vitro levels can conveniently be measured as described elsewhere herein, for example in bacterial cultures. Preferably, such increases are increases in extracellular levels of adenosine or 5′nucleotidase activity, for example as measured in vitro, for example in the supernatant of bacterial cultures.

An appropriate negative control strain might be a strain that does not contain a gene encoding a 5′nucleotidase or does not contain an active 5′nucleotidase enzyme (or does not have 5′nucleotidase activity as described elsewhere herein).

Because of the downstream uses of the strains which are selected by the methods of the invention, after adenosine producing strains are selected or isolated, other embodiments will involve the further steps of culturing or propagating or producing such strains, and optionally formulating said cultured or propagated or produced strains into a composition comprising said strain, e.g. a pharmaceutical or nutritional composition, e.g. as described elsewhere herein, or possibly storing such strains for future uses, for example through lyophilisation or freeze drying.

The selection steps of the methods of the invention (and indeed the therapeutic methods as described herein) will generally need to be carried out in an appropriate culture medium (or in vivo environment), which supports adenosine production. Preferred culture media (or in vivo environment) will contain an appropriate carbon source, which will support the production of adenosine by said strain, preferably together with an appropriate substrate for adenosine production by the 5′nucleotidase enzyme, e.g. AMP.

Although such assays can conveniently be carried out in vitro, another option would be to assess the strains in an appropriate in vivo assay, e.g. using an appropriate mouse model (see for example the mouse model and assays described in Example 5, where for example levels of adenosine in plasma can be assessed).

In embodiments where more than one bacterial strain is screened using the methods of the invention, the amount of generated adenosine or 5′nucleotidase activity can be quantified and the bacterial strain, e.g. the lactic acid bacterial strain, with the highest activity or level of the 5′-nucleotidase enzyme, or a sufficiently high activity or level of the 5′-nucleotidase enzyme, for example with activities or levels as described elsewhere herein, for example with activities or levels higher (preferably significantly higher) than DSM17938, or the strain which produces the highest amount or level of adenosine, or a sufficiently high amount or level of adenosine, for example with amounts or levels as described elsewhere herein, for example with amounts or levels higher (preferably significantly higher) than DSM17938, can be selected.

It is thus a yet further aspect of the invention to provide a method for the selection of bacterial strains, preferably lactic acid bacterial strains, effective in producing adenosine comprising:
a) screening lactic acid bacterial strains for the presence of a gene encoding a 5′-nucleotidase, e.g. a cell surface anchored 5′nucleotidase, and;
b) quantifying the activity of the 5′-nucleotidase enzyme and; optionally
   c) selecting the lactic acid bacterial strain which has the highest or sufficiently high activity of the 5′-nucleotidase enzyme.

Methods comprising certain steps as described herein also include, where appropriate, methods consisting of these steps.

As set out above, a bacterial strain, e.g. a lactic acid bacterial strain, selected, produced, obtained or obtainable by the methods of the invention, wherein said strain is capable of producing adenosine is a yet further aspect of the invention.

Any appropriate bacterial strain, e.g. probiotic bacterial strain, for example any probiotic bacteria, can be subjected to the selection methods of the invention and any appropriate bacterial strain, e.g. probiotic bacterial strain, which is capable of producing adenosine can be used in the methods or uses of the present invention, e.g. in the therapeutic methods or uses described herein.

Preferred bacterial strains are lactic acid bacteria, for example Lactobacillus or Bifidobacterium. Particularly preferred bacterial strains are Lactobacillus reuteri, in particular the strains Lactobacillus reuteri DSM 32846, DSM 32847, DSM 32848 and DSM 32849 that were deposited under the Budapest Treaty at the DSMZ-Deutsche Sammlung von Mikroorganismen und Zellkulturen GmbH (Mascheroder Weg 1b, Inhoffenstr. 7B, D-38124 Braunschweig) on Jul. 4, 2018 and Lactobacillus reuteri DSM 33198 that was deposited under the Budapest Treaty at the DSMZ-Deutsche Sammlung von Mikroorganismen und Zellkulturen GmbH (Mascheroder Weg 1b, Inhoffenstr. 7B, D-38124 Braunschweig) on Jul. 9, 2019. These bacterial strains or isolated bacterial strains (and other strains, e.g. L reuteri strains, in particular related L reuteri strains, with one or more of the characteristics, e.g. the ability to produce or induce the effects on the production of adenosine and/or 5′ nucleotidase activity, of one or more of these deposited strains) form preferred aspects of the invention and can for example be used to treat the diseases or conditions as described elsewhere herein or for any other uses as described herein. Another preferred strain for the therapeutic uses described herein is DSM 17938 (the depositary details of which are provided elsewhere herein). However, in some embodiments, the L. reuteri strain DSM 17938 is not used.

In some embodiments the bacterial strains used do not have the ability to produce histamine or significant levels of histamine. In some embodiments the bacterial strains used do not have the gene for histamine production (Histidine decarboxylase, hdcA (locus tag HMPREF0536_1230)). In some embodiments, the strain is not ATCC PTA 6475, ATCC PTA 4659, ATCC PTA 4965, ATCC 5289, ATCC 5290, CF15-6, CF4-6g, LMS11-1, LMS11-3, SR-11, SR-14, CF6-2a, CF2-a0, Me261, DSM20016, DSM 32229, DSM 32230, DSM 32231, DSM 32232 and/or DSM 32273.

Thus, a yet further aspect of the invention provides the use of such strains to treat or prevent one or more of the diseases as described herein.

It is another object of the invention to provide a method for improving the use and effect of the treatment of the specific disorders as described herein comprising maximizing, increasing or improving the activity of the 5′-nucleotidase of the selected strains, e.g. lactic acid bacterial strains, which will preferably lead to increased or improved production of adenosine. This can be achieved by culturing the bacteria in specific growth conditions to allow a high yield of adenosine (or a higher, e.g. significantly higher, yield of adenosine, e.g. compared to the yield observed in the absence of the specific growth condition). In one embodiment of the invention, the specific growth condition can be e.g. culturing the lactic acid bacteria in normal growth medium supplemented with an appropriate substrate for adenosine production by the 5′nucleotidase enzyme, e.g. AMP, or appropriate upstream components of AMP such as ADP and/or ATP.

In a further related embodiment of the invention, to maximize the activity of the 5′-nucleotidase, a higher concentration of bacteria, e.g. lactic acid bacteria, can be administered. An exemplary concentration might be 2 to 10 times higher than a conventional dose.

In a further related embodiment of the invention, the 5′-nucleotidase gene can be overexpressed or induced in the bacterial strains by appropriate methods, e.g. the insertion of a plasmid vector with the 5′-nucleotidase gene under the control of an appropriate promoter, e.g. an inducible promoter. Alternatively, the 5′-nucleotidase gene could be overexpressed by inserting one or more additional copies of the gene into the bacterial cell, for example insertion on the chromosome of the bacterial cell, under the control of an appropriate promoter, e.g. an inducible or the native promoter.

As described above, the strains of the invention, or strains selected by, produced, obtained, or obtainable by the methods of the invention have uses in therapy. Thus, a further aspect of the invention provides the adenosine producing strains of the invention, or strains which are selected, obtained or obtainable using the selection methods of the invention, for use in the production, e.g. local or systemic production, of adenosine. In preferred embodiments of the invention, said production of adenosine is used for the treatment of wounds, e.g. chronic wounds, or other diseases as described elsewhere herein, which will benefit from production or increased production, e.g. local or systemic production, of adenosine, etc.

The administration of the bacterial strains in the methods of treatment and uses of the invention is carried out in pharmaceutically, therapeutically, or physiologically effective amounts, to subjects (animals/mammals) in need of treatment. Thus, said methods and uses may involve the additional step of identifying a subject in need of treatment.

Treatment of disease or conditions in accordance with the present invention (for example treatment of pre-existing disease) includes cure of said disease or conditions, or any reduction or alleviation of disease (e.g. reduction in disease severity) or symptoms of disease.

The methods and uses of the present invention are suitable for prevention of diseases or conditions as well as treatment of diseases or conditions. Thus, prophylactic treatment is also encompassed by the invention. For this reason in the methods and uses of the present invention, treatment or therapy also includes prophylaxis or prevention where appropriate. Such preventative (or protective) aspects can conveniently be carried out on healthy or normal subjects as described herein, and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

A yet further aspect of the invention provides a strain or product for the therapeutic uses as defined elsewhere herein, wherein said use further comprises the administration of at least one further agent, e.g. a further therapeutic or nutritional agent. Exemplary agents might be substrate components which will increase or enhance the production of adenosine (e.g. a component which can increase or enhance the production of adenosine), or a source of such components.

Thus, in a yet further embodiment of the invention, where an adenosine producing bacteria is concerned, the administration of said strains or products further comprises the administration of a substrate component, e.g. AMP and/or a material or agent that produces this component. A preferred substrate would be AMP, or a source of AMP.

In such embodiments, the substrate component, or other additional component, could be added to the bacterial preparation directly before administration to the subject. Alternatively, it could be added to the bacterial preparation at the end of the manufacturing process, e.g. at the end of fermentation, after which the bacteria can be stored for future use, e.g. by lyophilisation or freeze-drying. Alternatively it can be administered separately as described below.

In such embodiments, the further therapeutic agent can be any further agent which is useful in the treatment of the disease in question.

Said further agents can be administered together with the strains or products of the invention (e.g. as a combined preparation) or can be administered separately. In addition, said further agents can be administered at the same time as the strains or products of the invention or at different time points, e.g. sequentially. Suitable administration regimes and timings can readily be determined by the skilled person depending on the further agent in question.

The present invention also provides a composition (or a combination product or kit) comprising:
(i) a bacterial strain, e.g. a lactic acid bacterial strain, selected, produced, obtained or obtainable by the selection method of the invention (or a bacterial strain capable of producing or inducing adenosine production as otherwise defined herein), wherein said bacterial strain has a gene encoding a cell surface anchored 5′-nucleotidase or an active 5′nucleotidase enzyme and is capable of producing adenosine; and
(ii) one or more substrate components or agents which will increase or enhance the production of adenosine, or a source of such components or agents.
   Exemplary components or agents are outlined above. A preferred component is AMP or a source of AMP.

The term “subject” as used herein includes mammals, in particular humans. In preferred embodiments of the invention, the selected strain, e.g. lactic acid bacterial strain, is administered to a human. Any subject, e.g. any human subject, e.g. adult or child or infant or elderly subject, suffering from said disease, or suspected to be suffering from said disease, or at risk from said disease is appropriate.

Thus, as described elsewhere herein, preferred subjects are those suffering from one or more of the diseases as outlined herein, or are subjects which are believed or suspected to be suffering from one or more of the diseases as outlined herein. As the therapeutic uses of the invention can also be used to prevent disease, appropriate subjects include those at risk of suffering from one or more of the diseases as outlined herein.

Other preferred diseases (and hence subjects) to be treated are diseases or conditions associated with adenosine deficiency (or subjects suffering from said diseases). For such diseases, exemplary subjects include subjects which have low, reduced e.g. significantly reduced (or abnormal) levels of adenosine or have adenosine deficiency, e.g. compared to levels in a normal or healthy subject, for example levels in a normal or healthy subject of the same, equivalent or comparable age.

In all such subjects, if appropriate or necessary, levels of adenosine in said subject can readily be measured using techniques readily available and known in the art. For example, levels of adenosine can readily be measured in serum/blood/plasma or saliva samples.

Any appropriate mode of administration can be used and can be readily selected depending on the disease in question. Conveniently said administration is oral and/or topical. Thus, the bacteria can be administered to the GI tract, GU tract (e.g. the vagina), oral cavity, skin or locally in the wound (or appropriate local administration for any other disease described herein), as desired or appropriate. The bacteria can be included in a probiotic topical formula or a food supplement or oil drops, as well as in a pharmaceutical formulation.

Appropriate doses of the strains, products and compositions of the invention as defined herein can readily be chosen depending on the disease (or condition) to be treated, the mode of administration and the formulation concerned.

For example, a dosage and administration regime is chosen such that the bacteria administered to the subject in accordance with the present invention can result in an increased production, e.g. local or systemic (e.g. plasma or blood) production, of adenosine and to give rise to the desired therapeutic effects or health benefits in wounds, e.g. chronic wounds, or in other diseases as described elsewhere herein. Thus, preferably said dosage is a therapeutically effective dosage which is appropriate for the type of mammal and condition being treated and is for example administered to a subject in need thereof. For example, daily doses, e.g. a single daily dose, of 10⁴ to 10¹², for example 10⁵ to 10⁹, or 10⁶ to 10⁸, or 10⁸ to 10¹⁰, or 10¹⁰ to 10¹² total CFUs of bacteria may be used.

The term “increase” or “enhance” or “higher” (or equivalent terms) as described herein includes any measurable increase or elevation when compared with an appropriate control. Appropriate controls would readily be identified by a person skilled in the art and might include levels when strains are not present, or levels in non-treated or placebo-treated subjects, or levels in a healthy or normal subject, e.g. an age-matched subject, or the same subject before treatment. Preferably the increase will be significant, for example clinically or statistically significant, for example with a probability value of ≤0.05.

Preferably such increases (and indeed other increases, improvements or positive effects as mentioned elsewhere herein) are measurable increases, etc., (as appropriate), more preferably they are significant increases, preferably clinically significant or statistically significant increases, for example with a probability value of ≤0.05, when compared to an appropriate control level or value (e.g. compared to an untreated or placebo treated subject or compared to a healthy or normal subject, or the same subject before treatment).

The term “decrease” or “reduce” (or equivalent terms) as described herein includes any measurable decrease or reduction when compared with an appropriate control. Appropriate controls would readily be identified by a person skilled in the art and might include levels when strains are not present, or levels in non-treated or placebo-treated subjects, or levels in a healthy or normal subject, e.g. an age-matched subject, or the same subject before treatment. Preferably the decrease or reduction will be significant, for example clinically or statistically significant, for example with a probability value of ≤0.05.

Thus, preferably such decreases (and indeed other decreases, reductions or negative effects as mentioned elsewhere herein) are measurable decreases, etc., (as appropriate), more preferably they are significant decreases, preferably clinically significant or statistically significant decreases, for example with a probability value of ≤0.05, when compared to an appropriate control level or value (e.g. compared to an untreated or placebo treated subject or compared to a healthy or normal subject, or the same subject before treatment).

As used throughout the entire application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms.

Lists “consisting of” various components and features as discussed herein can also refer to lists “comprising” the various components and features.

Other objects and advantages will be more fully apparent from the following non-limiting examples.

### EXAMPLES

### Example 1

### A Method for Identification of Strains Having a Gene Encoding a Cell Surface Located 5′-Nucleotidase

Cultivate the bacteria on MRS plates for 16 h at 37° C. in anaerobic atmosphere. Collect 10 bacterial colonies with a sterile plastic loop and suspended in 100 microlitres of sterile water (PCR quality). (Alternative: Prepare DNA from the bacterial culture using a suitable method).

Presence of the 5′-nucleotidase gene can be examined by PCR, e.g. by using PuReTaq Ready To Go PCR beads (GE HealthCare) and any of the primer pairs described below (0.4 mM of each). Bacterial suspension or DNA preparation (0.5 microliter) should be added to the PCR mix and the PCR reaction should be performed by running the program 95° C., 5 min; 30× (95° C., 30 s; 55° C., 30 s; 72° C., 30 s); 72°, 10 min. The PCR products could be separated and visualised by using standard agarose gel electrophoresis and the sequence determined by standard Sanger sequencing (using the forward primer used for the PCR).

The gene could be detected using any of the following primers:

Primer pair 1 (product size 233 bp)
(SEQ ID NO:2)
LrNuc1f: GGAACTTTGGGAAACCATGA
(SEQ ID NO:3)
LrNuc1r: CGGGCAACTTTACCATCACT
Primer pair 2 (product size 212 bp)
(SEQ ID NO:4)
LrNuc2f: TACTCGTGAAAATGCCGTTG
(SEQ ID NO:5)
LrNuc2r: GTGCCCCTGTCATTTCAACT
Primer pair 3 (product size 232 bp)
(SEQ ID NO:6)
LrNuc3f: AGCTTTACCAAATTGACCCTGA
(SEQ ID NO:7)
LrNuc3r: TTGATATTAGGCGCATCCTTTT

### Sequences

Gene encoding Cell surface anchored 5’-
nucleotidase, GenBank accession number:
AEI56270.1, LPXTG-motif cell wall anchor
domain protein [Lactobacillus reuteri SD2112]
(SEQ ID NO:8)
ATGAAGAATAATAGTTCAAAATATTGTTTATTGTTAGGGACAGCGCTGT
TAGGACTATATTTCCAAGCTAATAGTGTTCATGCGGATGCGACTGGTAT
CACAGCTAATGGAGAAACTACCCATAGCAATGTTACTCCAATGGTTCAG
ACTAATAAGGATGAGGCAAGTACACCGCAAACAACTACTGATTGGTCTG
ACCCGGCCAAATATCAAAGTGACATTCCAGTTCAGATTTTAGGAATCAA
TGACTTGCATGGTGGGTTAGAAACGACTGGATCAGCTACGATTGGAGAT
AAGACTTATTCGAATGCCGGAACAGTTGCACGCCTAGCTGGTAACCTTG
ATGCGGCGGAGGAAAGTTTTAAGAACGCTAATCCGACGGGAAGCTCAAT
TCGGGTAGAAGCCGGAGATATGGTTGGGGCTTCTCCAGCAAATTCTGCT
CTTCTCCAAGACGAATCAACCATGCATGCTTTAGACGCAATGCATTTTG
AAATAGGAACTTTGGGAAACCATGAGTTCGATGAAGGTTTAGCTGAGTA
TATGCGGATTGTTAATGGTGGTGAACCTACTAAACAATATAATGAAGCT
GAGATGGCCTATCCTCATGTGAAAACAGGGATTAATATCATTACTGCCA
ATGTTGTAAATAAATCTGATGGTCAAATCCCATTTGGAATGCAACCATA
CTTGATTAAAGAAATTCATACTAGTGATGGTAAAGTTGCCCGGATCGGA
TTTATTGGGATTGAAACTACTTCCCTACCAATTTTAACCTTATACGATA
ATTACAAAGATTATGATGTTTTAGACGAGGCTGAAACAATTGCAAAATA
TGATCAAATTTTACGCAAAAAAGGTGTTAACGCAATTGTAGTTCTTGCC
CATACAGGGGTTTCAACTGATAAAGATGGCAGCACTAAAGGTAATGCTG
TTGATATCATTAAGAAGCTTTACCAAATTGACCCTGATAATTCTGTCGA
CCTTTATATTGCTGGTCACTCCCACCAATATGCTAATGCTACTGTTGGA
AGTGTAAAATTAGTGCAAGCCATTTACACGGGTAAAGCTTACGATGATA
TTATCGGTTACATCGATCCAACAACTAATGATTTTGCGCCCAATAGTCT
CGTTTCACATGTCTTTCCGGTACTATCTGAAAAGGATGCGCCTAATATC
AAAACGGATGCAAATGTTACAGCAATTGTTGAAGATGCGAACAACCGAG
TAGCACCGATTATTAACAAGAAAATAGGGGAAGCTGCTACAACAGGCGA
TATTCTTGGACGACTTCATAATACTCCTACTCGTGAAAATGCCGTTGGT
GAATTAGTTGTCGATGGTCAATTATATGCCGCTCATAAAGTAGGCTTAC
CAGCTGATTTTGCGATGACTAATACAGGGGGCGTTCGTGCAGATCTGCA
TGTTAATCCTGATCGTTCCATTACATGGGGGAGTGCCCAAGCAGTTCAA
CCATTTGGTAATATTTTGCGGGTAGTTGAAATGACAGGGGCACAAATCG
TTGAAGCCTTGAATCAACAATACGACGAAGATCAAGCTTACTACTTACA
GATTTCCGGGCTACATTATACTTATACTGACCAAAACGATCCTAACCAA
CCATATAAGGTCGTTCAAGTTTATGACCAACATAATCAACCGCTTGATA
TGAATAAGACTTACAATGTTGTTATTAATGACTTTTTAGCAGGTGGCGG
AGATGGCTTTTCTGCATTTAAGGGTACTAAAGTTGTCGGGATTGTTGGT
CAAGATACAGACGCGTTTATTGACTATATTACTGATATGACTAATGATG
GTAAACCAATTACTGCGCCAACAATGAACCGTAAGATTTACTTGACTGC
TGAACAAGTAGCGAAGGCTGACTCAGATTCACAGTTACAAACAGGAACT
AATCAGAACACTCAAAACGATGCTAATTCCCAGACTGAAGGAAATCAGC
TTCAAGAAGTTCCGAGCCAACCGGTATCTCCAACAGTAACCTTGCCAAC
AACAGCTGGTCAACCCGCCGAAACTGTTACACTACATGCTCAATCTAAG
CAACAAACCGTAGCTGCTAATAATCAATTAATTAATTTGACGCCTACAT
CAATTAATGGCCAAAAACAAAAAGCAGCTGACCAGCAAGCAGCTTTACC
ACAAACTAGTAACGATGAAGATCTTGCATTACTTCTTCTCGGAAGTTCA
TTAATGGCAGCAACCGGATTGACAATTATTGATCGCAAGCGTAAACATG
CTTAA
Cell surface anchored 5’-nucleotidase protein,
GenBank accession number: AEI56270.1, LPXTG-
motif cell wall anchor domain protein
[Lactobacillus reuteri SD2112]
(SEQ ID NO:9)
MKNNSSKYCLLLGTALLGLYFQANSVHADATGITANGETTHSNVTPMVQ
TNKDEASTPQTTTDWSDPAKYQSDIPVQILGINDLHGGLETTGSATIGD
KTYSNAGTVARLAGNLDAAEESFKNANPTGSSIRVEAGDMVGASPANSA
LLQDESTMHALDAMHFEIGTLGNHEFDEGLAEYMRIVNGGEPTKQYNEA
EMAYPHVKTGINIITANVVNKSDGQIPFGMQPYLIKEIHTSDGKVARIG
FIGIETTSLPILTLYDNYKDYDVLDEAETIAKYDQILRKKGVNAIVVLA
HTGVSTDKDGSTKGNAVDIIKKLYQIDPDNSVDLYIAGHSHQYANATVG
SVKLVQAIYTGKAYDDIIGYIDPTTNDFAPNSLVSHVFPVLSEKDAPNI
KTDANVTAIVEDANNRVAPIINKKIGEAATTGDILGRLHNTPTRENAVG
ELVVDGQLYAAHKVGLPADFAMTNTGGVRADLHVNPDRSITWGSAQAVQ
PFGNILRVVEMTGAQIVEALNQQYDEDQAYYLQISGLHYTYTDQNDPNQ
PYKVVQVYDQHNQPLDMNKTYNVVINDFLAGGGDGFSAFKGTKVVGIVG
QDTDAFIDYITDMTNDGKPITAPTMNRKIYLTAEQVAKADSDSQLQTGT
NQNTQNDANSQTEGNQLQEVPSQPVSPTVTLPTTAGQPAETVTLHAQSK
QQTVAANNQLINLTPTSINGQKQKAADQQAALPQTSNDEDLALLLLGSS
LMAATGLTIIDRKRKHA

### Example 2

### Procedure for Analysing 5′-Nucleotidase Activity

The Crystal Chem 5′-Nucleotidase Assay Kit (Crystal Chem, Elk Grove Village, Ill., USA) was used for determining the 5′-nucleotidase activity of the bacterial cells and the fermentation supernatants. In short, the procedure was as follows.

In two steps, reagents CC1 and CC2 was added to the samples containing the bacteria or the supernatants. Reagent 1 contains AMP that was converted to adenosine by the 5′-nucleotidase enzyme from the bacteria. Adenosine was further hydrolysed into inosine and hypoxanthine by components in reagent 1. In the second step, hypoxanthine was converted to uric acid and hydrogen peroxide, which was used to generate a quinone dye that was measured kinetically at 550 nm in a spectrophotometer. The activity was determined by calculating the change in absorbance between 3 and 5 minutes and comparing with the value from a calibrator sample.

### Example 3

5′-Nucleotidase Activity in Lactobacillus reuteri Strains

Experimental data showing 5′-nucleotidase activity in Lactobacillus reuteri DSM 32846, DSM 32847, DSM 32848 and DSM 32849 as well as in Lactobacillus reuteri DSM 17938 (deposited under the Budapest Treaty at the DSMZ-Deutsche Sammlung von Mikroorganismen and Zellkulturen GmbH (Mascheroder Weg 1b, D-38124 Braunschweig) on 30 Jan. 2006) was generated using the method described in Example 2 above. The results are shown in FIG. 1.

### Example 4

### Selection of Strains

All new strains in Example 3 above, i.e. Lactobacillus reuteri DSM 32846, DSM 32847, DSM 32848 and DSM 32849 show 5′-nucleotidase activity in the bacterial supernatant. Analysis was carried out on the supernatant of bacterial cultures with the concentration of 10⁹ bacteria per ml.

Lactobacillus reuteri DSM 32846, DSM 32847, DSM 32848 and DSM 32849 have been developed/evolved for improved properties. DSM 32846 and DSM 32847 have been made to evolve to be more tolerant to bile acids and thereby survive in larger numbers in the GI-tract. DSM 32848 and DSM 32849 have evolved to adhere better to mucus, with the aim to colonize better in the GI-tract and thereby function better according to the present invention.

All strains Lactobacillus reuteri DSM 32846, DSM 32847, DSM 32848 and DSM 32849 are selected. Preferred strains are DSM 32846, DSM 32847 and DSM 32849.

### Example 5

### Effect of Strains on Levels of Adenosine in Plasma

### Materials and Methods

Mice. Wild type (WT) C57BL/6J mice (6-8 week-old) were purchased from Jackson Laboratories and allowed to acclimatize for 2 weeks before experimentation. All mice were housed in the specific pathogen free animal facility at the University of Texas Health Science Center at Houston. This study was carried out in accordance with the recommendations of the Guide for the Care and Use of Laboratory Animals (NIH), the Institutional Animal Care and Use Committee (IACUC). The protocol was approved by the IACUC (protocol numbers: AWC-14-056 and 17-0045).

Lactobacillus reuteri preparation. Human breast milk-derived Lactobacillus reuteri DSM 17938 (LR) was provided by BioGaia AB (Stockholm, Sweden). LR was anaerobically cultured in deMan-Rogosa-Sharpe (MRS) medium at 37° C. for 24 h, then plated in MRS agar at specific serial dilutions and grown anaerobically at 37° C. for 48-72 h to count colonies for generating the standard curve of bacterial colony-forming units (CFU)/mL grown on MRS agar vs. absorbance at 600 nm at known concentrations. Quantitative analysis of bacteria in the culture media on CFU/mL then was calculated by comparing absorbance at 600 nm of cultures by using the standard curve.

Experimental Design. Newborn mice were generated by breeding of C57BL/6J female to male mice and were remained with their dams. We set up three experimental groups: WT+LR (n=8 mice): orally feeding each newborn mouse by gavage with freshly grown LR in cultured MRS media (10⁷ CFU/day, 100 μL), daily, starting from 8 days of age (d8) to d21 before weaning; WT+MRS control (n=8 mice): orally feeding each newborn mouse by gavage with an identical volume of MRS media, daily, starting from d8 to d21 before weaning; Finally, WT no treatment control (n=8 mice): newborn mice stayed with their dam without feeding either LR or MRS. Mice were euthanized on d22 to collect blood and cecal contents. The plasma and cecal contents were stored immediately at −80° C. freezer for further plasma metabolomics analysis, and stool microbiota analysis.

Plasma global metabolomic analysis. Plasma metabolites of mice with WT no treatment, WT+MRS and WT+LR were measured by Metabolon Inc. (www.metabolon.com). A total of 688 compounds of known identity (named biochemicals) in plasma were detected by a non-targeted metabolomic analysis platform including UPLC-MS/MS and GC/MS, respectively. The global metabolomics profile data including normalized scale (intensity), pathway heat map, and fold change of WT+LR/WT no treatment or WT+LR/WT+MRS were reported by Metabolon Inc.

Statistical analysis. Group comparisons of the intensity scales of adenosine and other metabolites were performed by using Two-Way ANOVA corrected for multiple comparisons with Bonferroni posttests by using Prism 4.0 (GraphPad Software, San Diego, Calif., USA), P-values<0.05 were considered significant.

### Results

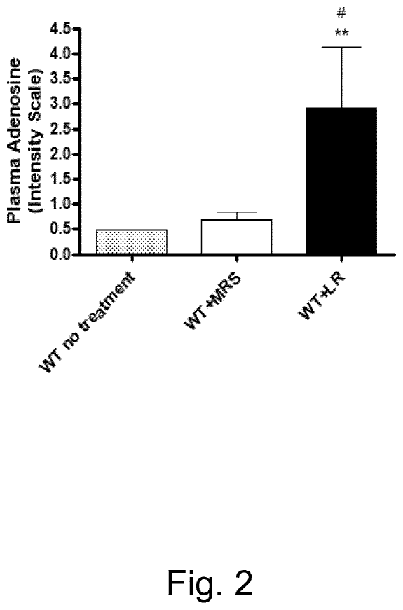

Metabolomics analysis detected plasma adenosine. Oral feeding Lactobacillus reuteri DSM 17938 daily to healthy breastfed mice significantly increased the intensity scales of plasma adenosine compared to mice fed MRS control group (p<0.05) or mice no treatment group (p<0.01) (FIG. 2). The fold change of WT+LR/WT no treatment=5.95 and the fold change of WT+LR/WT+MRS=4.4. There were no significant difference between groups of WT+MRS vs. WT no treatment.

### Example 6

5′-Nucleotidase Activity in Lactobacillus reuteri Strains

Experimental data showing 5′-nucleotidase activity in Lactobacillus reuteri DSM 33198 was generated using the method described in Example 2 above. The results are shown in FIG. 3A where they have been normalized in relation to the 5′-nucleotidase activity of DSM17938 (DSM17938 activity=1) from the same experiment. The same type of normalization in relation to the 5′-nucleotidase activity of DSM17938 (DSM17938 activity=1) has also been done on the results from FIG. 1 and this is presented in FIG. 3B, making it easy to compare the fold-change in 5′-nucleotidase activity in relation to DSM17938 for the different bacterial strains.

### Example 7

### Selection of Strains

The new strain in Example 6 above, i.e. Lactobacillus reuteri DSM 33198 show high 5′-nucleotidase activity in the bacterial supernatant. Analysis was carried out on the supernatant of the bacterial culture with the concentration of 10⁹ bacteria per ml.

Lactobacillus reuteri DSM 33198 has been developed for improved properties. The L. reuteri strain DSM 33198 has been modified in a multi-step selection process including a repeated freeze-drying procedure to allow it to be more tolerant and give a higher survival in the production process than its native isolate.

Lactobacillus reuteri DSM 33198 is selected.

1.-20. (canceled)

21. A method of treating or preventing a disease or condition associated with a deficiency or reduction in adenosine in a subject in need thereof, comprising administering to the subject a therapeutically effective amount of a Lactobacillus reuteri strain that is capable of producing or inducing the production of adenosine in the subject,
wherein administration of said strain leads to increased 5′-nucleotidase activity and/or levels of adenosine production as compared to the levels upon administration of Lactobacillus reuteri DSM 17938.

22. The method of claim 21, wherein administration of said strain leads to increases of up to 5 fold in 5′-nucleotidase activity and/or levels of adenosine production as compared to the levels upon administration of Lactobacillus reuteri DSM 17938; or increases of at least 1.5 fold, of at least 2 fold, or of at least 5 fold.

23. The method of claim 21, wherein said strain has a 5′-nucleotidase activity of greater than 8 units/L, and/or produces or induces the production of adenosine in the subject at a level of greater than 8 μmol L⁻¹ min⁻¹.

24. The method of claim 21, wherein the method comprises treating a wound.

25. The method of claim 21, wherein the method comprises administering the strain topically to skin or hair.

26. The method of claim 21, wherein the disease or condition associated with a deficiency or reduction in adenosine comprises an inflammatory condition.

27. The method of claim 21, wherein the administration is topical or oral, and wherein the method comprises treating intestinal wounds, restoring skin, anti-ageing treatments, promoting hair thickening or hair growth, treating or preventing hair loss, treating or preventing inflammatory processes in the gastrointestinal tract, genitourinary tract, oral cavity, in the lungs and/or airways, or on skin.

28. The method of claim 21, wherein said strain is Lactobacillus reuteri DSM 32846, DSM 32847, DSM 32849 and/or DSM 33198.

29. The method of claim 28, wherein said strain is Lactobacillus reuteri DSM 32846.

30. The method of claim 21, further comprising administering at least one further substrate component or agent that increases the production of adenosine in the subject or is a source of adenosine, wherein the at least one further agent is administered as a combined preparation with the Lactobacillus reuteri strain or is administered separately.

31. The method of claim 30, wherein said at least one further agent comprises AMP (adenosine monophosphate) or is a source of AMP.

32. A bacterial strain, wherein said strain is Lactobacillus reuteri DSM 32846, DSM 32847, DSM 32849 or DSM 33198.

33. The bacterial strain of claim 32, wherein said strain is Lactobacillus reuteri DSM 32846.

34. A method of isolating a lactic acid bacterial strain capable of producing adenosine, wherein said strain is Lactobacillus reuteri and wherein said method comprises:
a) screening the Lactobacillus reuteri strain for the presence of a gene encoding a 5′-nucleotidase and/or
screening the Lactobacillus reuteri strain or its supernatant, for the presence of an active 5′-nucleotidase enzyme; and
b) detecting the presence of a gene encoding a 5′-nucleotidase and/or an active 5′-nucleotidase enzyme;
c) isolating the Lactobacillus reuteri strain, wherein the strain is capable of producing adenosine, at a level which is increased as compared to Lactobacillus reuteri DSM 17938; and optionally
d) culturing or propagating the Lactobacillus reuteri strain.

35. A Lactobacillus reuteri strain obtainable by the method of claim 34.

36. The Lactobacillus reuteri strain of claim 35, wherein said strain:
(i) has a 5′-nucleotidase activity of greater than 8 units/L and/or produces adenosine at a level of greater than 8 μmol L-1 min −1; and/or
(ii) can give rise to increases of up to 5 fold in 5′-nucleotidase activity and/or levels of adenosine production as compared to the levels with Lactobacillus reuteri DSM 17938; or increases of at least 1.5 fold, of at least 2 fold, or of at least 5 fold.

37. A combination product for increasing the production of adenosine in a subject comprising:
(i) a first Lactobacillus reuteri strain that is capable of producing or inducing the production of adenosine, wherein said strain is the strain of claim 32; and
(ii) a further source of adenosine or one or more substrate components or agents, that increases the production or induction of adenosine by said Lactobacillus reuteri strain, wherein said substrate component is AMP or a source of AMP.

38. A method of producing or inducing the production of adenosine in a subject comprising administering an effective amount of a Lactobacillus reuteri strain that is capable of producing or inducing the production of adenosine to said subject, wherein said strain is the strain of claim 32.


SEQUENCE LISTING (9 sequences)

SEQ ID NO: 1 - protein, 5 aa, Artificial Sequence
  Motif
  MISC_FEATURE (3)..(3): any amino acid
LPXTG

SEQ ID NO: 2 - DNA, 20 bp, Artificial Sequence
  Primer LrNuc1f
ggaactttgggaaaccatga

SEQ ID NO: 3 - DNA, 20 bp, Artificial Sequence
  Primer LrNuc1r
cgggcaactttaccatcact

SEQ ID NO: 4 - DNA, 20 bp, Artificial Sequence
  Primer LrNuc2f
tactcgtgaaaatgccgttg

SEQ ID NO: 5 - DNA, 20 bp, Artificial Sequence
  Primer LrNuc2r
gtgcccctgtcatttcaact

SEQ ID NO: 6 - DNA, 22 bp, Artificial Sequence
  Primer LrNuc3f
agctttaccaaattgaccctga

SEQ ID NO: 7 - DNA, 22 bp, Artificial Sequence
  Primer LrNuc3r
ttgatattaggcgcatcctttt

SEQ ID NO: 8 - DNA, 2259 bp, Lactobacillus reuteri SD112
atgaagaataatagttcaaaatattgtttattgttagggacagcgctgttaggactatatttccaagctaatagtgttcatgcggatgcgactggtatcacagctaatggagaaactacccatagcaatgttactccaatggttcagactaataaggatgaggcaagtacaccgcaaacaactactgattggtctgacccggccaaatatcaaagtgacattccagttcagattttaggaatcaatgacttgcatggtgggttagaaacgactggatcagctacgattggagataagacttattcgaatgccggaacagttgcacgcctagctggtaaccttgatgcggcggaggaaagttttaagaacgctaatccgacgggaagctcaattcgggtagaagccggagatatggttggggcttctccagcaaattctgctcttctccaagacgaatcaaccatgcatgctttagacgcaatgcattttgaaataggaactttgggaaaccatgagttcgatgaaggtttagctgagtatatgcggattgttaatggtggtgaacctactaaacaatataatgaagctgagatggcctatcctcatgtgaaaacagggattaatatcattactgccaatgttgtaaataaatctgatggtcaaatcccatttggaatgcaaccatacttgattaaagaaattcatactagtgatggtaaagttgcccggatcggatttattgggattgaaactacttccctaccaattttaaccttatacgataattacaaagattatgatgttttagacgaggctgaaacaattgcaaaatatgatcaaattttacgcaaaaaaggtgttaacgcaattgtagttcttgcccatacaggggtttcaactgataaagatggcagcactaaaggtaatgctgttgatatcattaagaagctttaccaaattgaccctgataattctgtcgacctttatattgctggtcactcccaccaatatgctaatgctactgttggaagtgtaaaattagtgcaagccatttacacgggtaaagcttacgatgatattatcggttacatcgatccaacaactaatgattttgcgcccaatagtctcgtttcacatgtctttccggtactatctgaaaaggatgcgcctaatatcaaaacggatgcaaatgttacagcaattgttgaagatgcgaacaaccgagtagcaccgattattaacaagaaaataggggaagctgctacaacaggcgatattcttggacgacttcataatactcctactcgtgaaaatgccgttggtgaattagttgtcgatggtcaattatatgccgctcataaagtaggcttaccagctgattttgcgatgactaatacagggggcgttcgtgcagatctgcatgttaatcctgatcgttccattacatgggggagtgcccaagcagttcaaccatttggtaatattttgcgggtagttgaaatgacaggggcacaaatcgttgaagccttgaatcaacaatacgacgaagatcaagcttactacttacagatttccgggctacattatacttatactgaccaaaacgatcctaaccaaccatataaggtcgttcaagtttatgaccaacataatcaaccgcttgatatgaataagacttacaatgttgttattaatgactttttagcaggtggcggagatggcttttctgcatttaagggtactaaagttgtcgggattgttggtcaagatacagacgcgtttattgactatattactgatatgactaatgatggtaaaccaattactgcgccaacaatgaaccgtaagatttacttgactgctgaacaagtagcgaaggctgactcagattcacagttacaaacaggaactaatcagaacactcaaaacgatgctaattcccagactgaaggaaatcagcttcaagaagttccgagccaaccggtatctccaacagtaaccttgccaacaacagctggtcaacccgccgaaactgttacactacatgctcaatctaagcaacaaaccgtagctgctaataatcaattaattaatttgacgcctacatcaattaatggccaaaaacaaaaagcagctgaccagcaagcagctttaccacaaactagtaacgatgaagatcttgcattacttcttctcggaagttcattaatggcagcaaccggattgacaattattgatcgcaagcgtaaacatgcttaa

SEQ ID NO: 9 - protein, 752 aa, Lactobacillus reuteri SD112
MKNNSSKYCLLLGTALLGLYFQANSVHADATGITANGETTHSNVTPMVQTNKDEASTPQTTTDWSDPAKYQSDIPVQILGINDLHGGLETTGSATIGDKTYSNAGTVARLAGNLDAAEESFKNANPTGSSIRVEAGDMVGASPANSALLQDESTMHALDAMHFEIGTLGNHEFDEGLAEYMRIVNGGEPTKQYNEAEMAYPHVKTGINIITANVVNKSDGQIPFGMQPYLIKEIHTSDGKVARIGFIGIETTSLPILTLYDNYKDYDVLDEAETIAKYDQILRKKGVNAIVVLAHTGVSTDKDGSTKGNAVDIIKKLYQIDPDNSVDLYIAGHSHQYANATVGSVKLVQAIYTGKAYDDIIGYIDPTTNDFAPNSLVSHVFPVLSEKDAPNIKTDANVTAIVEDANNRVAPIINKKIGEAATTGDILGRLHNTPTRENAVGELVVDGQLYAAHKVGLPADFAMTNTGGVRADLHVNPDRSITWGSAQAVQPFGNILRVVEMTGAQIVEALNQQYDEDQAYYLQISGLHYTYTDQNDPNQPYKVVQVYDQHNQPLDMNKTYNVVINDFLAGGGDGFSAFKGTKVVGIVGQDTDAFIDYITDMTNDGKPITAPTMNRKIYLTAEQVAKADSDSQLQTGTNQNTQNDANSQTEGNQLQEVPSQPVSPTVTLPTTAGQPAETVTLHAQSKQQTVAANNQLINLTPTSINGQKQKAADQQAALPQTSNDEDLALLLLGSSLMAATGLTIIDRKRKHA